(19) United States
(12) Patent Application Publication
(10) Pub. No.: US 2011/0077305 A1
(43) Pub. Date: Mar. 31, 2011

(54) 5-LIPOXYGENASE INHIBITORS

(76) Inventor: William Paul Jackson, Twickenham (GB)

(21) Appl. No.: 12/995,298

(22) PCT Filed: Jun. 1, 2009
(86) PCT No.: PCT/EP2009/003906

§ 371 Date: Nov. 30, 2010

(30) Foreign Application Priority Data
Jun. 2, 2008 (GB) 0810011.7

(51) Int. Cl. A61K 31/18; C07C 311/16; A61P 35/00

(52) U.S. Cl. 514/604; 564/92

(57) ABSTRACT

The use of compounds of the formula (I) Ar¹-L¹-Ar²-L²-C(R³)(R⁴)N(OR¹)C(═Y)—R² (I) where Y is selected from O or S; R¹ is H, a salt or readily hydrolysable substituent; R² is selected from H or CH₃, CH₂F, CF₂H or CF₃; R³ and R⁴ are selected independently from H, C 1-4 alkyl or alkenyl, CF₃, CH₂F, CF₂H and F, with the proviso that if either R³ or R⁴ is H, then the other is not H; L¹ is a linker group; L² is a linker group comprising an optionally substituted or unsubstituted unsaturated branched or straight chain alkyl group; Ar¹ is an optionally substituted or unsubstituted aryl or heterocyclic group; and Ar² is an optionally substituted or unsubstituted aryl or heterocyclic group, in the treatment of 5-lipoxygenase mediated cancer provide improved therapies due to the effective inhibition of 5-lipoxygenase and long duration of activity in vivo after oral administration.

### FIELD OF THE INVENTION

This invention pertains generally to the field of biologically active compounds, and more specifically to the use of certain hydroxamic acid compounds for the inhibition of 5-lipoxygenase (5-LO or 5-LOX), both in vitro and in vivo, and for the prophylaxis or treatment of cancers in which 5-lipoxygenase is implicated. The present invention also pertains to certain classes of sulphonamide-containing hydroxamic acid compounds.

### BACKGROUND OF THE INVENTION

The 5-lypoxygenase pathway has for some time been known to play a role in some inflammation related diseases, such as asthma, psoriasis and rheumatoid arthritis.

The 5-lipoxygenase pathway is one of several metabolic pathways identified arachidonic acid (AA) in humans. The 5-lypoxygenase pathway is believed to convert AA into pro-inflammatory metabolites, 5-hydroxyeicosatetraenoic acid (5-HETE), 5-oxo-eicosatetraenoic acids (5-oxoETEs), leukatrienes B4 (LTB4), and cysteinyl leukotrienes (LTC4, LTD4 and LTE4), as well as anti-inflammatory metabolite, lipoxin A4 (LXA4) and lipoxin B4 (LXB4). The major metabolic enzymes are 5-lipoxygenase (5-LOX), 5-lipoxygenase activating protein (FLAP), leukatrienes A4 hydrolase (LTA4H), and leukatrienes C4 synthase (LTC4S).

In view of the role of 5-lypoxygenase in asthma, psoriasis and rheumatoid arthritis, there have been numerous attempts to find pharmaceutically acceptable lipoxygenase inhibitors. Among these, several hydroxamic acid derivatives have been described.

Zileuton (Zyflo™) is an approved 5-lypoxygenase inhibitor for the treatment of asthma. Zileuton, which is described for example in WO-A-94/2629, is a hydroxyl urea having a benzothienylethyl group and is used in racemic form.

U.S. Pat. No. 5,714,633 discloses further hydroxyureas for 5-lipoxygenase inhibitory activity, produced by Abbott Laboratories.

U.S. Pat. No. 4,977,188 and U.S. Pat. No. 4,988,733 disclose a series of ‘normal’ and ‘reverse’ hydroxamic acid derivatives as inhibitors of 5-lipoxygenase. The second of these patents refers to a series of compounds in which L is a trans-olefin and X is oxygen. However, in neither of the patents, is a sulphonamide linking group disclosed. The preferred compounds in this case have an oxygen linking the two aryl units, e.g.

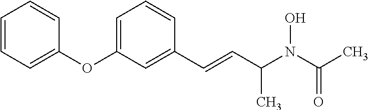

Other biologically active hydroxamic acid derivatives have been described in the prior art, but not as 5-lipoxygenase inhibitors. For example, there are several disclosures of hydroxamic acid derivatives as HDAC (histone deacetylase) inhibitors.

U.S. Pat. No. 5,534,654 describes a novel class of hydroxamic acid compounds capable of cell growth and vascularisation inhibition. In particular, it discloses a sulphonamide-containing hydroxamic acid of the structure below, known as Oxamflatin, which is used extensively in biological studies.

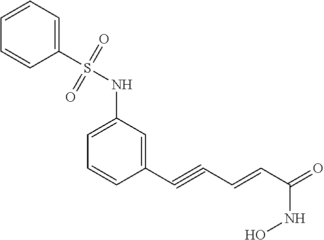

### (2E)-N-hydroxy-5-{3-[(phenylsulfonyl)amino]phenyl}pent-2-en-4-ynamide

Oxamflatin

WO-A-01/18171 describes a class of HDAC inhibiting hydroxamic acid and specifically discloses a single sulphonamide linked molecule.

WO-A-011/38322 (Delorme et al) relates to compounds for the inhibition of histone deacetylase (HDAC) enzymatic activity and methods for treating cell proliferation diseases and conditions. The compounds described therein according to the general formula are all ‘normal’ hydroxamic acids, substituted amides and derivatives thereof. Among exemplified compounds are a number of hydroxamic acid compounds containing sulphonamide linker groups, such as the molecule below.

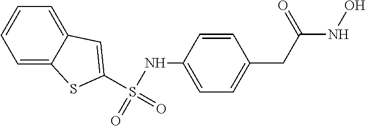

More recently, in US 2004/0077726A1, a series of sulphonamide linked carbamic acid compounds as HDAC inhibitors for the treatment of cancer and psoriasis have been described. Among the described compounds are ones in which the sulphonamide linkage has been “reversed”, as illustrated in the molecule below which is currently undergoing clinical trials.

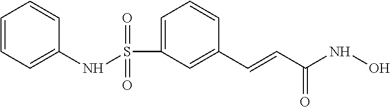

WO 2007/039403 (Atlanta Pharma) discloses a class of ‘normal’ hydroxamic acids having N-sulphonyl pyrrole functionalities, which compounds are described as being crystalline and having HDAC inhibitory activity.

WO-A-2005/061448 is concerned with methods of treating vascular diseases, and particularly with the treatment of aneurysm, using known compounds such as amiloride and oxamflatin as well as some novel sulphonamide-containing hydroxamic acid derivatives. Among the hydroxamic acid derivatives falling within the scope of the general formula disclosed are ‘reversed’ hydroxamic acids (i.e. —N(OH)—COR). Whilst most specified compounds were ‘normal’ hydroxamic acids, one specifically stated (although not exemplified) ‘reverse’ hydroxamic acid structure is:

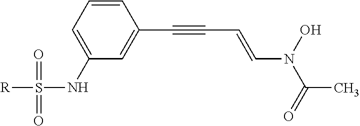

More recently, 5-lipoxygenase and the 5-LO pathway have been implicated in the mechanism of action of, and 5-LO has been found to be overexpressed in, certain human cancers.

A drawback for many of the 5-lipoxygenase inhibitors previously described in connection with the application to cancer is that the therapeutic duration of action is often insufficient and some compounds have been found to be toxic. No effective treatment for cancer comprising a 5-lipoxygenase inhibitor has been approved. There has been no disclosure of a ‘reverse’ hydroxamic acid as a 5-lipoxygenase inhibitor for treating cancer, particularly having a sulfonamide group.

There remains a need for well tolerated, longer acting and more efficacious inhibitors of 5-lipoxygenase for the treatment of cancer.

### PROBLEM TO BE SOLVED BY THE INVENTION

There is therefore a continuing problem in providing effective treatments for cancer in providing 5-lipoxygenase inhibitors for the effective treatment of certain cancers.

It is an object of this invention to provide a pharmaceutical treatment for cancers implicating 5-lipoxygenase.

It is a further object of the invention to provide novel compounds that provide the desired biological activity and pharmaceutical stability to enable effective treatment of certain cancers, e.g. anti-proliferative compounds acting via 5-lipoxygenase pathway inhibition.

Thus, it is a further object of the invention to provide compounds which are potent inhibitors of the 5-lipoxygenase pathway and, in particular, of 5-lipoxygenase itself.

Such molecules, especially in the treatment and/or prophylaxis of certain cancers, desirably have one or more of the following properties and/or effects: (a) easily gain access to and act upon tumour cells; (b) down-regulate 5-lipoxygenase activity; (c) inhibit tumour cell proliferation; (d) promote tumour cell apoptosis; (e) inhibit tumour growth; and, (f) complement the activity of traditional chemotherapeutic agents.

### SUMMARY OF THE INVENTION

In accordance with a first aspect of the invention, there is provided a compound for use in the inhibition of 5-lipoxygenase for the treatment or prophylaxis of cancer, which compound is defined according to Formula I

Ar¹-L¹-Ar²-L²-C(R³)(R⁴)N(OR¹)C(═Y)—R² (I)

where

Y is selected from O or S

R¹ is H, a salt or readily hydrolysable substituent;

R² is selected from H or CH₃, CH₂F, CF₂H or CF₃;

R³ and R⁴ are selected independently from H, C1-4 alkyl or alkenyl, CF₃, CH₂F, CF₂H and F, with the proviso that if either R³ or R⁴ is H, then the other is not H;

L¹ is a linker group;

L² is a linker group comprising an optionally substituted or unsubstituted unsaturated branched or straight chain alkyl group;

Ar¹ is an optionally substituted or unsubstituted aryl or heterocyclic group; and

Ar² is an optionally substituted or unsubstituted aryl or heterocyclic group.

In a second aspect of the invention, there is provided a compound of the formula (II)

Ar¹-L¹-Ar²-L²-C(R³)(R⁴)N(OR¹)C(═Y)—R² (II)

where

Y is selected from O or S;

R¹ is H, a salt or readily hydrolysable substituent;

R² is selected from H or CH₃, CH₂F, CF₂H or CF₃;

R³ and R⁴ are selected independently from H, C1-4 alkyl or alkenyl, CF₃, CH₂F, CF₂H and F, with the proviso that if either R³ or R⁴ is H, then the other is not H;

L¹ is NHSO₂, SO₂NH, NHCONH, SO₂N—CH₂—, -(L³)ₙ-SO₂NH-(L⁴)ₘ- or (L³)ₙ-NHSO₂-(L⁴)ₘ where n and m are 0 or 1 and L³ and L⁴ are selected from CH₂ and branched or straight-chain C2-4 alkyl or alkenyl, or —SO₂— provided that where L¹ is —SO₂— then Ar¹ or Ar² is bound to L¹ via a ring nitrogen;

L² is an unsaturated C2-6 optionally substituted or unsubstituted branched or straight chain alkyl group;

Ar¹ is an optionally substituted or unsubstituted aryl or heterocyclic group; and

Ar² is an optionally substituted or unsubstituted aryl or heterocyclic group.

In a third aspect of the invention, there is provided a compound according to Formula II above for use in therapy.

In a fourth aspect of the invention, there is provided a compound according to Formula II above for use in the treatment or prophylaxis of cancer.

In a fifth aspect of the invention, there is provided a compound according to Formula II or Formula I above for use in the inhibition of 5-lipoxygenase in the therapeutic treatment of cancer.

In a sixth aspect of the invention, there is provided a compound according to Formula II above for use in the treatment of 5-lipoxygenase mediated cancer or cancers implicating 5-lipoxygenase by inhibition of 5-lipoxygenase.

In a seventh aspect of the invention, there is provided a pharmaceutical formulation comprising the compound according to Formula II above and a pharmaceutically acceptable excipient.

In an eighth aspect of the invention, there is provided a use of a compound according to Formula I or Formula II above in the manufacture of a medicament for the treatment or prophylaxis of cancers in which 5-lipoxygenase is implicated by inhibition of 5-lipoxygenase.

In a ninth aspect of the invention, there is provided a use of an inhibitor of 5-lipoxygenase and/or an inhibitor of HDAC in the manufacture of a medicament indicated for the treatment of cancer by combination therapy using an inhibitor of 5-lipoxygenase in combination with an inhibitor of HDAC.

In a tenth aspect of the invention, there is provided a use of a 5-lipoxygenase inhibitor in the manufacture of a medicament indicated for the treatment of cancer in combination with a 15-lipoxygenase activator.

In an eleventh aspect of the invention, there is provided a method for the treatment or prophylaxis of cancer in the human or animal body comprising administering to a patient in need thereof a therapeutically effective amount of 5-lipoxygenase inhibitor in order to disrupt 5-lipoxygenase activity, the 5-lipoxygenase inhibitor being selected from compounds according to Formula I or Formula II above.

### ADVANTAGES OF THE INVENTION

The invention provides compounds for use in the therapeutic treatment of cancer by inhibition of 5-lipoxygenase, which compounds are effective inhibitors of 5-lipoxygenase whilst having excellent pharmaceutical stability. The invention further provides novel compounds having good biological profile and potent 5-lipoxygenase inhibitory activity whilst being metabolically stable and having a relatively long duration of action in vivo. The compounds defined herein provide improved treatments of 5-lipoxygenase implicated cancers, such as esophageal cancer, bladder cancer, prostate cancer and pancreatic cancer.

### DETAILED DESCRIPTION OF THE INVENTION

The classes of compounds used according to the invention and the novel class of compounds defined herein are ‘reverse’ hydroxamic acid derivatives which are defined below. By ‘reverse’ hydroxamic acids, it is meant that the hydroxamic acid derivative function —N(OR)C(O)R′ is formed from a ‘simple’ acid and a ‘complex’ hydroxylamine whilst a ‘normal’ hydroxamic acid will have the formula —C(O)NR(OR′) which is derived from a ‘complex’ acid and a ‘simple’ hydroxylamine. By simple acid, it is meant a low molecular weight carboxylic acid with minimal substituents, such as acetic acid, trifluoroacetic acid or formic acid. By simple hydroxylamine, it is meant a hydroxylamine with a low molecular weight and simple substituents, such as hydroxylamine with an NH or N-lower alkyl/cycloalkyl group. ‘Complex’ acids and hydroxylamines will have more substantial and complex substituents. Accordingly, in a ‘reverse’ hydroxamic acid, the hydroxylamine portion will have a significantly higher molecular weight than the acid portion. In the case of sulphonamide-containing reverse hydroxamic acids, for example, the sulphonamide group will form part of the complex hydroxylamine portion of the molecule.

As mentioned above, the compounds according the first aspect of the invention are for use in the treatment of cancer by inhibition of 5-lipoxygenase, by which it is meant inhibition of the 5-lipoxygenase pathway, which comprises down regulating the activity of 5-lipoxygenase, and which may be by inhibition of 5-lipoxygenase activating protein, leukotrienes A4 hydrolase and C4 synthase and 5-lipoxygenase enzyme itself. Inhibition of the 5-lipoxygenase enzyme itself is preferred. The compounds according to the first aspect are further for use in the treatment or prophylaxis of cancers in which one or more 5-lipoxygenase pathway metabolic enzymes, preferably 5-lipoxygenase, is implicated or that are 5-lipoxygenase mediated (or p53-based tumours), which treatment or prophylaxis should be effected by inhibition of 5-lipoxygenase (or associated metabolic enzymes).

Compounds according to the first aspect of the invention, as stated above may be defined according to the following formula I.

Ar¹-L¹-Ar²-L²-C(R³)(R⁴)N(OR¹)C(═Y)—R² (I)

where

Y is selected from O or S

R¹ is H, a salt or readily hydrolysable substituent, such as a hydrolysable ester, a —CH₂-ester group or a —CH₂—O—PO(OH)₂ group;

R² is selected from H or CH₃, CH₂F, CF₂H or CF₃;

R³ and R⁴ are selected independently from H, C1-4 alkyl or alkenyl, CF₃, CH₂F, CF₂H and F, with the proviso that if either R³ or R⁴ is H, then the other is not H;

L¹ is a linker group, which may be any suitable linker but is preferably selected from O, S, NHSO₂, SO₂NH;

L² is a linker group comprising an optionally substituted or unsubstituted unsaturated branched or straight chain alkyl group;

Ar¹ is an aryl or heterocyclic group, which may, for example, be an optionally substituted or unsubstituted phenyl or 5 or 6 membered heterocycle having 1-4 heteroatoms;

Ar² is an aryl or heterocyclic group, which may, for example, be an optionally substituted or unsubstituted phenyl or a 5 or 6 membered heterocycle having 1-4 heteroatoms and optionally either or both of Ar¹ and Ar² incorporate L¹ within its structure.

The compounds defined by formula I as set out above are now discussed in more detail, with further examples of each of the features of the compounds defined.

Any aryl-containing group may form Ar¹ and Ar², which may be bound to the adjacent linker group via a substituent group, but is preferably directly bonded via an aryl carbon or heteroatom. The groups Ar¹ and Ar² may independently be any suitable aryl group and may independently represent aromatic hydrocarbon and fused aromatic hydrocarbon ring structures, aromatic and non-aromatic heterocyclic groups, each of which may be substituted or unsubstituted. For example, Ar¹ and Ar² may independently represent an optionally substituted or unsubstituted C6-10 aryl group or an optionally substituted or unsubstituted aromatic or non-aromatic 5 to 10 membered heterocyclic group. The C6-10 aryl group may be selected from, for example, a phenyl or naphthyl group or tetrahydronaphthyl group, which may be substituted or unsubstituted. The 5 to 10 membered heterocyclic group may be an aromatic heterocyclic group, for example 5 or 6 membered ring structures comprising at least one ring heteroatom and optionally two, three or four heteroatoms, which may for example be selected from O, S and N. Examples of such heterocyclic groups include pyridyl, pyrazinyl, pyrimidinyl, pyridazinyl, furanyl, thiaphenyl, thienyl, imidazolyl, pyrazolidinyl, pyrrolyl, oxadiazolyl, oxazyl, isoxazyl, thiadiazolyl, thiazolyl, 1,2,3-triazolyl, 1,2,4-triazolyl, tetrazolyl and pyrazolyl. Alternatively, the 5 to 10 membered heterocycle is non-aromatic, i.e. saturated or partially unsaturated, C5-10 carbocyclic ring having one or more, e.g. 2, 3 or 4, heteroatoms, which, for example, may be selected from O, S or N. Examples of such heterocylces include piperidinyl, piperazinyl, morpholinyl, pyrrolidinyl, tetrahydrofuranyl, imidazolidinyl, thiazolidinyl, 1,4-dioxanyl and 1,3-dioxanyl. Each of the above heterocycles may be substituted or unsubstituted. Optionally, the heterocycle may form a fused ring system, such as a quinolinyl, benzothienyl, benzofuranyl or benzothiazolyl.

The aryl, aromatic heterocycle and non-aromatic heterocyclic groups may optionally be substituted or unsubstituted, as mentioned above. If substituted, they may be substituted with any suitable substituents, which may be selected from, for example, C1-10 alkyl, C2-10 alkenyl, C2-10 alkynyl, C1-10 alkoxy, C1-10 thioalkoxy, hydroxyl, C1-10 hydroxyalkyl, halo, C1-10 haloalkyl, amino, C1-10 alkylamino, di(C1-10 alkyl)amino, amido, nitro, cyano, (C1-10 alkyl)carbonyloxy, (C1-10 alkoxy)carbonyl, (C1-10 alkyl)carbonyl, (C1-10 alkyl)thiocarbonyl, (C1-10 alkyl)sulfonylamino, aminosulfonyl, (C1-10 alkyl)sulfinyl, (C1-10 alkyl)sulfonyl or C1-10 alkyl substituted by amino, C1-10 alkoxy, C1-10 alkylamino or di(C1-10 alkyl)amino. Preferably the substituents may independently be selected from C1-4 alkyl, C1-4 alkoxy, amino, C1-4 alkylamino, di(C1-4 alkyl)amino), halo, C1-4alkyl substituted by one, two or three chlorine or fluorine atoms, (C1-4 alkoxy)carbonyl or C1-4 alkyl substituted by amino, C1-2 alkoxy, C1-2 alkylamino, di(C1-2 alkyl)amino, cyano, amido or nitro. Still more preferably the substituents may be selected from methyl, ethyl, methoxy, ethoxy, dimethylamino, bromo, chloro, fluoro, trifluoromethyl, difluoromethyl, fluoromethyl, methoxymethyl, ethoxymethyl, aminomethyl, methylaminomethyl or dimethylaminomethyl.

Preferably, at least Ar² and more preferably both Ar¹ and Ar² are selected from aromatic aryl or heterocyclic systems. More preferably Ar¹ and Ar² independently represent:
i) napthyl, tetrahydronapthyl, furanyl, thiophenyl, thienyl or pyridyl, any of which is optionally unsubstituted or substituted by one or more of the substituents identified above; or
ii) phenyl optionally unsubstituted or substituted by one or more (e.g. two, three or four) of the substituents identified above.

More preferably, at least Ar² and still more preferably both Ar¹ and Ar² are phenyl groups which independently are optionally unsubstituted or substituted as defined above, but preferably with one or more bromo, chloro or fluoro substituent. Optionally, Ar² may be a pyridyl group optionally unsubstituted or substituted as defined above.

Where Ar² comprises a phenyl ring, it may be linked to L¹ and L² by any two atoms but preferably meta (1,3 arrangement) or para (1,4 arrangement). Where Ar¹ comprises a substituted phenyl ring, the substitution arrangement is such that at least one substituent is meta (1,3) or para (1,4) to the bond with L¹.

L¹ is a linker group. The linker group may be any suitable group for linking Ar¹ to Ar² and for example may be selected from a single bond, —C(R⁵)═N—, —N═C(R⁵)—, C(R⁵)(R⁶)—NR⁷—, —NR⁷—C(R⁵)(R⁶)—, —CO—NR⁵—, NR⁵—CO—, NR⁵—CO—NR⁶, —SO₂—NR⁵—, —NR⁵—SO₂—, -(L³)ₘ-SO₂—NR⁵-(L⁴)ₙ-, -(L³)ₘ-NR⁵—SO₂-(L⁴)ₙ-, —C(R⁵)(R⁶)—O—, —O—C(R⁵)(R⁶)—, —C(R⁵)(R⁶)—S—, —S—C(R⁵)(R⁶)—, —CONH—, NHCO—, —CO—, —SO—, —SO₂—, O, S, —[C(R⁵)R⁶]ₚ- (especially —CH₂—), —NH—SO₂— or —SO₂NH—, wherein R⁵, R⁶ and R⁷ each independently represents hydrogen, C1-6 alkyl, C6-10 aryl or a 5 to 10 membered heterocyclic group, L³ and L⁴ each independently represent a linking group corresponding to those defined for R⁵ which is optionally substituted or unsubstituted, m and n independently represent 0 or 1 the sum of which is preferably 1 or 2, and p is an integer of from 1 to 4. Preferably, L¹ is selected from O, S, NHSO₂ or SO₂NH or sulfonamide derivative. More preferably, L¹ is a sulfonamide or derivative. Optionally the —NH group of the sulfonamide forms a part of an adjacent aryl group Ar¹ or Ar². For example, the —NH group may form part of the ring structure of a pyrrole or other nitrogen containing heterocycle and form part of the linker group L¹ by being directly bound to an SO₂ group.

L² is an optionally substituted or unsubstituted unsaturated branched or straight chain alkyl group and comprises one or more alkene and/or alkyne moieties. The straight chain preferably comprises C2-C6, more preferably C2-C4 and most preferably is a C2 group. Preferably, L² is an ethenyl or ethynyl group. Most preferably, L² comprises an (E) —CH═CH— group.

Preferably, the compound according to the invention is in enantiomerically pure form, especially with reference to the carbon of L² adjacent the reverse hydroxyamide moiety, and more preferably is the S isomer.

One preferred class of compounds is that according to formula I in which Ar¹ is phenyl optionally substituted by one or more substituents independently selected from C1-4 alkyl (which may be substituted by one or more halogen atoms) and halogen; Ar² is a 1,3 or 1,4 phenylene group; L¹ is O; L² is an ethenyl group, preferably the trans (E) stereoisomer; R¹ is H; R² is H or C1-4 alkyl; R³ is H or C1-4 alkyl; and R⁴ is C1-4 alkyl. Preferably, the compounds of this class are in high purity enantiomeric form, preferably the S enantiomer. A particularly preferred member of this class of compounds is (E) N{1(S)-methyl-3-[3-(4-fluorophenoxy)phenyl]prop-2-en-1-yl}acetohydroxamic acid (as disclosed in EP-A-0351214).

A more preferred class of compounds according the first aspect of the invention is a class of novel compounds described and claimed herein in accordance with a second aspect of the invention, which compounds are defined according to the formula II:

Ar¹-L¹-Ar²-L²-C(R³)(R⁴)N(OR¹)C(═Y)—R² (II)

where

Y is selected from O or S;

R¹ is H, a salt or readily hydrolysable substituent, such as a hydrolysable ester, a —CH₂-ester group or a —CH₂—O—PO(OH)₂ group;

R² is selected from H or CH₃, CH₂F, CF₂H or CF₃;

R³ and R⁴ are selected independently from H, C1-4 alkyl or alkenyl, CH₂F, CF₂H, CF₃ and F, with the proviso that both R³ and R⁴ are not H;

L¹ is NHSO₂, SO₂NH, NHCONH, SO₂N—CH₂—, -(L³)ₙ-SO₂NH-(L⁴)ₘ- or (L³)ₙ-NHSO₂-(L⁴)ₘ where n and m are 0 or 1 and L³ and L⁴ are selected from CH₂ and branched or straight-chain C2-4 alkyl or alkenyl, or —SO₂— provided that where L¹ is —SO₂— then Ar¹ or Ar² is bound to L¹ via a ring nitrogen;

L² is an unsaturated C2-6, preferably C2-4, optionally substituted or unsubstituted branched or straight chain alkyl group;

Ar¹ is an aryl or heterocyclic group, which may, for example, be an optionally substituted or unsubstituted phenyl or 5 or 6 membered heterocycle having 1-4 heteroatoms;

Ar² is an aryl or heterocyclic group, which may, for example, be an optionally substituted or unsubstituted phenyl or a 5 or 6 membered heterocycle having 1-4 heteroatoms and optionally either or both of Ar¹ and Ar² incorporate L¹ within its structure.

Ar¹ and Ar² may be any group as defined for Ar¹ and Ar² for formula I above and the preferred groups. They may each independently be optionally substituted phenyl groups or heterocycle groups, e.g. Ar² may be a thienyl, pyrrolyl or furyl group whilst Ar¹ may be a pyridyl group.

L² is preferably a C2 alkenyl or alkynyl group, more preferably ethenyl and still more preferably trans (E) ethenyl.

C(R³)(R⁴) is preferably a —CH(CH₃) group.

R¹ which may be H, a salt or readily hydrolysable substituent, such as a hydrolysable ester, a —CH₂-ester group or a —CH₂—O—PO(OH)₂ group, is preferably H.

Y is preferably O.

R² is preferably CH₃.

Optionally, L¹ is NHCONH, SO₂NCH₂—, —SO₂—, provided that where L¹ is —SO₂— then Ar¹ or Ar² is bound to L¹ via a ring nitrogen; or L¹ is -(L³)ₙ-SO₂NH-(L⁴)ₘ- or (L³)ₙ-NHSO₂-(L⁴)ₘ where n and m are independently 0 or 1 provided that (m+n) is not 0 and L³ and L⁴ are selected from CH₂ and branched or straight-chain C2-4 alkyl or alkenyl. Compounds according to this class represent novel compounds.

The compounds according to this aspect of the invention have the benefit of being potent inhibition of 5-lipoxygenase and, at the same time, have a long duration of activity in vivo after oral administration.

In a preferred embodiment of this aspect of the invention are compounds according to the formula III

Ph¹-L¹-Ph²-L²-C(R³)(R⁴)N(OR¹)C(═Y)—R² (III)

where

Y is selected from O or S;

R¹ is H, a salt or readily hydrolysable substituent, such as a hydrolysable ester, a —CH₂-ester group or a —CH₂—O—PO(OH)₂ group;

R² is selected from H or CH₃, CH₂F, CF₂H or CF₃;

R³ and R⁴ are selected independently from H, C1-4 alkyl or alkenyl, CH₂F, CF₂H, CF₃ and F, with the proviso that both R³ and R⁴ are not H;

L¹ is NHSO₂ or SO₂NH (or optionally, L¹ is NHCONH, SO₂NCH₂—, —SO₂—, provided that where L¹ is —SO₂— then Ar¹ or Ar² is bound to L¹ via a ring nitrogen; or L¹ is -(L³)ₙ-SO₂NH-(L⁴)ₘ- or (L³)ₙ-NHSO₂-(L⁴)ₘ where n and m are independently 0 or 1 provided that (m+n) is not 0 and L³ and L⁴ are selected from CH₂ and branched or straight-chain C2-4 alkyl or alkenyl);

L² is an unsaturated C2-4 optionally substituted or unsubstituted branched or straight chain alkyl group;

Ph¹ is an optionally substituted or unsubstituted phenyl group

Ph² is an optionally substituted or unsubstituted phenyl group

When substituted, the phenyl groups Ph¹ and Ph² may be substituted with any of the substituents mentioned with respect to Ar¹ and Ar² above, but are preferably substituted with halides, e.g. one or more of each of F, Cl, Br or I, but preferably one or more of Br, Cl and/or F. The substitution arrangement of Ph¹ (where there is at least one substituent) is that at least one substituent relative to the bond to L¹ is in a 1,3 or 1,4 phenyl substitution pattern, but preferably 1,4. The substitution arrangement of Ph² of L² relative to L¹ is preferably 1,3 or 1,4, but more preferably 1,3. Ph² may be substituted or unsubstituted (aside from the linking groups L¹ and L²), but is preferably unsubstituted.

The following structures are particularly preferred compounds according to this aspect of the invention.

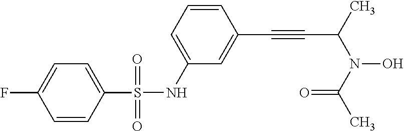

### N-[3-(3-{[(4-fluorophenyl)sulfonyl]amino}phenyl)-1-methylprop-2-yn-1-yl]-N-hydroxyacetamide

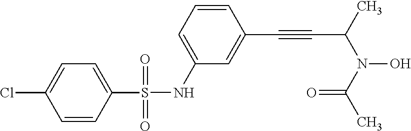

### N-[3-(3-{[(4-chlorophenyl)sulfonyl]amino}phenyl)-1-methylprop-2-yn-1-yl]-N-hydroxyacetamide

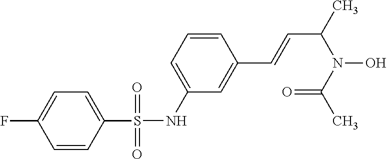

### N-[(2E)-3-(3-{[(4-fluorophenyl)sulfonyl]amino}phenyl)-1-methylprop-2-en-1-yl]-N-hydroxyacetamide

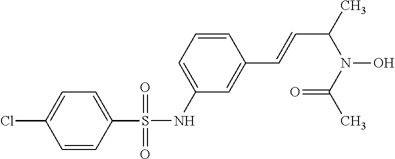

### N-[(2E)-3-(3-{[(4-chlorophenyl)sulfonyl]amino}phenyl)-1-methylprop-2-en-1-yl]-N-hydroxyacetamide

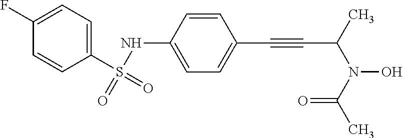

### N-[3-(4-{[(4-fluorophenyl)sulfonyl]amino}phenyl)-1-methylprop-2-yn-1-yl]-N-hydroxyacetamide

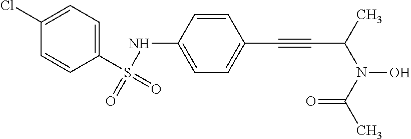

### N-[3-(4-{[(4-chlorophenyl)sulfonyl]amino}phenyl)-1-methylprop-2-yn-1-yl]-N-hydroxyacetamide

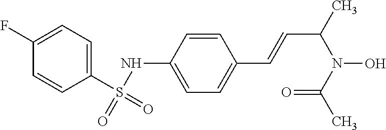

### N-[(2E)-3-(4-{[(4-fluorophenyl)sulfonyl]amino}phenyl)-1-methylprop-2-en-1-yl]-N-hydroxyacetamide

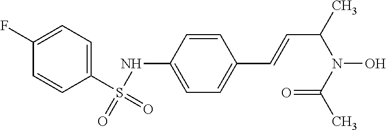

### N-[(2E)-3-(4-{[(4-fluorophenyl)sulfonyl]amino}phenyl)-1-methylprop-2-en-1-yl]-N-hydroxyacetamide

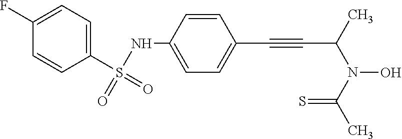

### N-[3-(4-{[(4-fluorophenyl)sulfonyl]amino}phenyl)-1-methylprop-2-yn-1-yl]-N-hydroxyethanethioamide

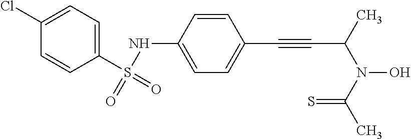

### N-[3-(4-{[(4-chlorophenyl)sulfonyl]amino}phenyl)-1-methylprop-2-yn-1-yl]-N-hydroxyethanethioamide

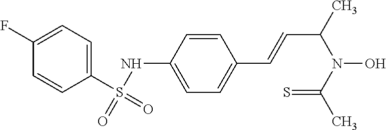

### N-[(2E)-3-(4-{[(4-fluorophenyl)sulfonyl]amino}phenyl)-1-methylprop-2-en-1-yl]-N-hydroxyethanethioamide

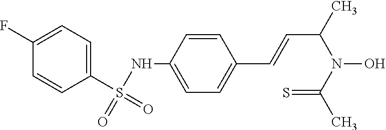

### N-[(2E)-3-(4-{[(4-fluorophenyl)sulfonyl]amino}phenyl)-1-methylprop-2-en-1-yl]-N-hydroxyethanethioamide

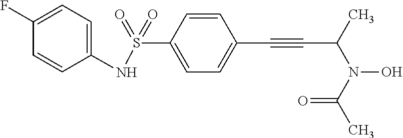

### N-(3-{4-[(4-fluorophenyl)sulfamoyl]phenyl}-1-methylprop-2-yn-1-yl)-N-hydroxyacetamide

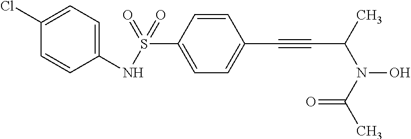

### (3-{4-[(4-chlorophenyl)sulfamoyl]phenyl}-1-methylprop-2-yn-1-yl)-N-hydroxyacetamide

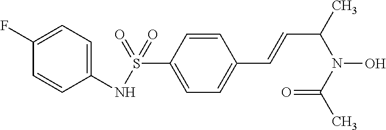

### N-[(2E)-3-{4-[(4-fluorophenyl)sulfamoyl]phenyl}-1-methylprop-2-en-1-yl]-N-hydroxyacetamide

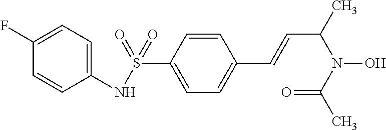

### N-[(2E)-3-{4-[(4-fluorophenyl)sulfamoyl]phenyl}-1-methylprop-2-en-1-yl]-N-hydroxyacetamide

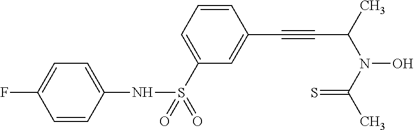

### N-(3-{3-[(4-fluorophenyl)sulfamoyl]phenyl}-1-methylprop-2-yn-1-yl)-N-hydroxyethanethioamide

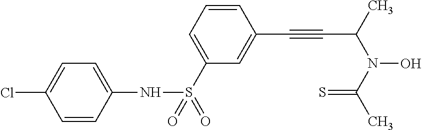

### N-(3-{3-[(4-chlorophenyl)sulfamoyl]phenyl}-1-methylprop-2-yn-1-yl)-N-hydroxyethanethioamide

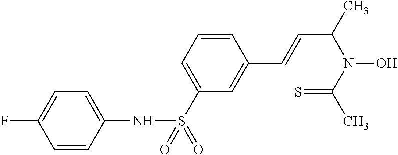

### N-[(2E)-3-{3-[(4-fluorophenyl)sulfamoyl]phenyl}-1-methylprop-2-en-1-yl]-N-hydroxyethanethioamide

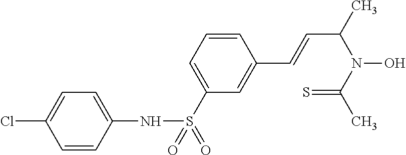

### N-[(2E)-3-{3-[(4-chlorophenyl)sulfamoyl]phenyl}-1-methylprop-2-en-1-yl]-N-hydroxyethanethioamide

### N-[(2E)-3-{3-[(4-chlorophenyl)sulfamoyl]phenyl}-1-methylprop-2-en-1-yl]-N-hydroxyacetamide

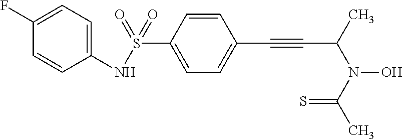

### N-(3-{4-[(4-fluorophenyl)sulfamoyl]phenyl}-1-methylprop-2-yn-1-yl)-N-hydroxyethanethioamide

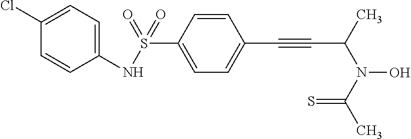

### N-(3-{4-[(4-chlorophenyl)sulfamoyl]phenyl}-1-methylprop-2-yn-1-yl)-N-hydroxyethanethioamide

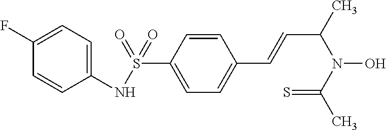

### N-[(2E)-3-{4-[(4-fluorophenyl)sulfamoyl]phenyl}-1-methylprop-2-en-1-yl]-N-hydroxyethanethioamide

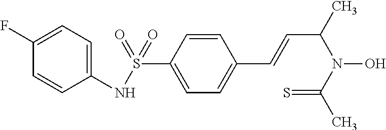

### N-[(2E)-3-{4-[(4-fluorophenyl)sulfamoyl]phenyl}-1-methylprop-2-en-1-yl]-N-hydroxyethanethioamide

The compounds may be in the racemic form or, more preferably, in the (R) or (S) optically active forms.

In the above general formulae, the R¹ group is defined as being a hydrogen, in order to form an N—OH group, or a derivative, bio-precursor or pro-drug thereof. The R¹ group may therefore be a metal ion such as Ca⁺ or Na⁺ (or other suitable counter-ion) in order to form a salt of the N—O⁻ group. Alternatively, the R¹ group may be any suitable pro-drug or protective group which may be readily cleaved in vivo, e.g. by hydrolysis. Suitable such groups may be provided when R¹ represents, for example, a —CH₂-ester group or a —CH₂—O—PO(OH)₂ or when R¹ represents the acid portion of an ester group with the O of —N—OH. Such bio-precursors or pro-drugs may further be such as to comprise any suitable substituent as the R¹ group which can be converted in vivo to the free compound or physiologically acceptable salt thereof.

The compounds described above may also be used as pharmaceutically acceptable salts thereof. A pharmaceutically acceptable salt, as referred to herein, is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, acorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium), alkali earth metal (e.g. calcium or magnesium), zinc and iron hydroxides and organic bases such as C1-6 alkyl amines, aralkyl amines or heterocyclic amines. An example of a primary amine salt can be the cyclohexylammonium salt, a suitable secondary amine salt may be the piperidine salt and a tertiary amine salt may be the triethylamine salt.

The compounds of the invention may contain one or more chiral centre, although in the preferred embodiment of the invention it contains a single chiral centre. For the avoidance of doubt, the chemical structures depicted here are intended to embrace all stereoisomers of the compounds shown, including racemic and non-racemic mixtures and pure enantiomers and/or diastereomers. The compounds of the invention and used in accordance with the invention may be in racemic form or, preferably, in optically active form. For example, in the preferred embodiment according to formula II in which the compounds have a single chiral centre, the compounds used may include an R enantiomer in substantially pure form, an S enantiomer in substantially pure form or enantiomeric mixtures which contain an excess of the R enantiomer or an excess of the S enantiomer. Preferably, the compound has a chiral centre at the alpha position to the hydroxylamine moiety, which is in enantiomerically high purity and is preferably the S enantiomer.

The compounds of formula II and III may be prepared by, or in adapted form, procedures known and previously described in the literature. A compound of formula II or III may be prepared, for example (in a non limiting sequence) according to Scheme I below or the methods described in the examples.

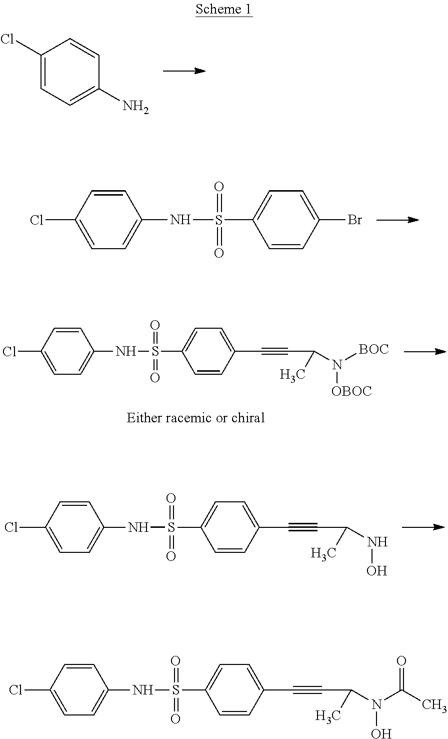

The olefinic compounds may be prepared by substituting the appropriate olefin, e.g. butenol. The thiohydroxamic acids may be prepared from the hydroxylamine using methods outlined in Synthesis, 1971, 110-130 and Heteroatom Chemistry, 13, 2002, 169-194.

The broader class of compounds of formula I may be prepared by known methods, such as that above and those set out in EP-A-0299761 and EP-A-0351214. In a further aspect of the invention, there is provided a process for the manufacture of a compound according to formula II, said process being derived from the above exemplified method.

In accordance with a further aspect of the invention, there is provided a compound as defined in formula II above for use in the inhibition of 5-lipoxygenase in the therapeutic treatment of cancer.

In accordance with a further aspect of the invention, the compounds defined above are for use in the treatment and/or prophylaxis of 5-lipoxygenase mediated cancer and cancers in which 5-lipoxygenase is implicated. The treatment or prophylaxis is effected by administering to a patient in need thereof a therapeutically effective amount of any one of the compounds defined above. The condition and/or symptoms associated with the condition can thereby be improved.

The compounds of the invention may be effective inhibitors of any metabolic enzymes of the 5-lipoxygenase pathway, but are preferably are effective by direct inhibition of 5-lipoxygenase.

Cancers that may be treated according to the present invention include, for example, breast cancer, colon cancer, colorectal cancer, esophageal cancer, glioma, leukemia; lung cancer including non-small cell lung cancer, prostate cancer, pancreatic cancer, bladder cancer, brain cancers, thoracic cancer, melanoma, ovarian cancer, cervical cancer, testicular cancer and renal cancer as well as further epithelial cell derived cancers. Further conditions include Rubinstein-Taybi syndrome, acute promyelocytic leukaemia, acute myelogenous leukaemia and non-hodgekins lymphoma.

Cancers that find particular beneficial effect are likely to be those that are epithelial cell derived. Examples of such cancers are discussed in U.S. Pat. No. 6,071,949.

Without being bound by theory, it is believed that structural elements of the compounds of the invention that contribute to the beneficial effect in the treatment of cancer include: an unsaturated element of L², the presence of a substituted methylene alpha to the hydroxamic acid nitrogen and a reverse hydroxamic acid in order individually to enhance metabolic stability; and a reverse hydroxamic acid and methyl-based substituent on the hydroxamic acid carbonyl in order individually to enhance the binding to iron to effect 5-lipoxygenase inhibitory activity.

The compounds of the invention may, in some circumstances, be advantageously used in combination with other therapies and in particular with other drug therapies. Optionally the compounds described herein can be co-administered together with or sequentially with a second drug. The combination therapy resulting may have a synergistic benefit.

For example, in the treatment of cancer, the compounds described herein may optionally be co-administered with, for example, platinum drugs such as cis-platin, alkylating agents such as chlorambucil or temozolomide, topoisomerase inhibitors such as the Topo II inhibitor etoposide, kinase cdk inhibitors such as flavopiridol or roscovitine, bcr-abl kinase inhibitors such as Glivec®, hsp 90 inhibitors, telomerase inhibitors and/or carbamylating agents. Other chemotherapeutic or antineoplastic agents that may be co-administered with compounds described herein include, for example, mitoxantron, Vinca alkaloids such as vincristine and vinblastine, anthracycline antibiotics, taxanes such as paclitaxel, antifolates such as methotrexate and camptothecins such as irinotecan.

Preferably, in order to efficiently target proliferative cancer cells via apoptosis the compounds of the invention may be co-administered with a topoisomerase II inhibitor such as etoposide or with roscovotine.

The compounds described herein may be co-administered with other 5-lipoxygenase inhibitors (or inhibitors of 12-lipoxygenase).

The compounds described herein may be co-administered with other therapies such as, for example, biologics such as TNF alpha inhibitors Remicade® and Enbrel, or thalidomide.

For medical use, the amount required of a compound defined above (hereinafter referred to as the active ingredient) to achieve a therapeutic effect will very much depend upon the particular compound used, the route of administration and with the particular disorder or disease being treated or prevented. Nevertheless, a suitable dose of a compound of formula (I) or (II) or a physiologically acceptable salt or derivative thereof for a mammal suffering from or at risk of suffering from any condition as described herein before (i.e. mediated by or implicating HDAC) is in the range 0.1 μg to 500 mg of the compound per kg of bodyweight. In the case of systemic administration, a suitable dose may be 0.5 mg to 500 mg per kg bodyweight, preferably 0.5 mg to 50 mg, for example from 5 mg to 25 mg per kg, administered, for example, three times daily. In the case of topical administration, a suitable dose is in the range of 0.1 ng to 100 μg per kg bodyweight, typically about 0.1 μg/kg.

In the case of oral dosing, a preferred dosage may be, for example, 1 mg to 10 mg of compound per kg bodyweight, more preferably 1 mg to 5 mg per kg, for example 1 mg to 2 mg per kg.

Whilst it may be possible for the compounds defined above to be administered alone, it is preferable to present it as a pharmaceutical formulation, which is provided as a further aspect of the present invention, and which comprises a compound of the formula I or formula II or a pharmaceutically acceptable salt thereof and a pharmaceutically acceptable excipient. Typically, the active ingredient comprises from 0.1 to 99.9% by weight of the formulation. Unit doses may comprise from 0.1 mg to 1 g of the active ingredient. For topical administration, the active ingredient preferably constitutes from 1% to 2% by weight of the formulation, but may constituted as much as 10% w/w. Formulations suitable for nasal or buccal administration typically comprise from 0.1 to 20% w/w, for example 2% w/w of active ingredient.

The pharmaceutical acceptable carrier or excipient should be compatible with other ingredients of the formulation and not detrimental to the recipient.

Formulations according to the invention include those in a form suitable for oral, pulmonary, ophthalmic, rectal parenteral (including subcutaneous, intramuscular and intravenous), intra-articular, topical, nasal or buccal administration. Formulations suitable for oral administration may be in the form of discrete units such as capsules, tablets or lozenges, each containing a predetermined amount of active ingredient; in the form of a powder or granules; in the form of a solution or suspension in an aqueous or non-aqueous liquid; or in the form of an oil-in-water or water-in-oil emulsion. Formulations for rectal administration may be in the form of a suppository incorporating the active ingredient, or in the form of an enema. Formulations for parenteral administration typically comprise a sterile aqueous preparation of the active ingredient, which is preferably isotonic with the blood of the recipient. Formulations for intra-articular administration may be in the form of a sterile aqueous preparation of the active ingredient, which may be in microcrystalline form. Formulations suitable for topical administration include liquid and semi liquid preparations such as liniments, lotions and applications; oil-in-water and water-in-oil emulsions such as creams, ointments and pastes; and solutions and suspensions such as drops.

In addition to the aforementioned ingredients, the formulations of the invention may include one or more additional ingredients such as diluents, buffers, flavouring agents, binders, surface active agents, thickeners, lubricants, preservatives, emulsifying agents and the like.

According to a further aspect of the invention, a 5-lipoxygenase inhibitor may be used in the treatment of cancer in combination with an HDAC inhibitor. This may be a mechanism for synergistic effect by inhibition via two different cancer drug targets, in order to cause apoptosis in the cancer cells, and produce an effective treatment. It is believed that this combined therapy will find particular application in prostate and pancreatic cancer.

A 5-lipoxygenase inhibitor and/or an HDAC inhibitor may therefore be used to manufacture a medicament indicated for the treatment of cancers (e.g. those referred to above in respect of 5-lipoxygenase alone) by combination therapy using an inhibitor of 5-lipoxygenase and an inhibitor of HDAC.

Preferably, the 5-lipoxygenase inhibitor and/or the HDAC inhibitor is selected from compounds comprising or derived from hydroxamic acids, more preferably ‘reverse’ hydroxamic acids. More preferably, the 5-lipoxygenase inhibitor and/or the HDAC inhibitor are selected from compounds defined according to Formula IV

(Ar¹-L¹)ₖ-Ar²-L²-C(R³)(R⁴)-Q N(OR¹)C(═Y)—R² (IV)

where

Ar¹ is an optionally substituted or unsubstituted aryl or heterocyclic group;

Ar² is an optionally substituted or unsubstituted aryl or heterocyclic group;

R³ and R⁴ are selected independently from H, C1-4 alkyl or alkenyl, CF₃, CH₂F, CF₂H and F, with the proviso that if either R³ or R⁴ is H, then the other is not H;

L¹ is a linker group, preferably as defined above and most preferably SO₂NH or NHSO₂;

L² is a linker group comprising an optionally substituted or unsubstituted unsaturated branched or straight chain alkyl group;

Q is represented by the formula —(C═Y)ₚN(OR¹)—(C═Y)_{q}R², where one of p and q is 0 and the other is 1

Y is selected from O or S

R¹ is H, a salt or readily hydrolysable substituent; and

R² is selected from H or CH₃, CH₂F, CF₂H or CF₃;

Still more preferably, the 5-lipoxygenase inhibitor and/or the HDAC inhibitor are selected from compounds defined according to any of Formulae I, II or III above. Preferably, both the 5-lipoxygenase inhibitor and the HDAC inhibitor are selected from compounds falling within the scope of any one of Formulae I, II, III or IV as defined herein.

By ‘combined therapy’ or ‘combination’ therapy or treatment with 5-lipoxygenase and an HDAC inhibitor (or other drug regime) it is meant treatment of a patient with both a 5 lipoxygenase inhibitor and an HDAC inhibitor, which may be by co-administration, sequential administration or by treating a patient with both therapies separately (e.g. by administering a 5-lipoxygenase inhibitor to a patient already receiving HDAC inhibitory treatment or vice versa).

Optionally, the 5-lipoxygenase inhibitor and the HDAC inhibitor are the same compound, by which it is meant that the compound has a dual mechanism of action on cancer cells (e.g. prostate cancer)—inhibition of 5-lipoxygenase and inhibition of HDAC.

This embodiment of the invention enables a more advantageous window of efficacy due to dual action, longevity and synergistic effect (leading to reduced effective toxicity issues).

Preferably the dual action compound is a hydroxylamine and more preferably is a compound according to any one of Formulae I, II, III or IV as defined above.

In a further aspect of the invention, a 5-lipoxygenase inhibitor may be used in the treatment of cancer in combination with a cPLA₂ inhibitor, preferably a cPLA₂-alpha inhibitor. This aspect of the invention finds particular application in the treatment of prostate cancer. Preferably, the 5-lipoxygenase is a hydroxylamine, more preferably a sulfonamine-containing ‘reverse’ hydroxylamine, and may be any compound according to the Formulae I, II, III and IV as defined above. The cPLA₂-alpha inhibitor may be any pharmaceutically acceptable cPLA₂-alpha inhibitor, but is preferably a compound selected from those cPLA₂-alpha inhibitors defined in WO-A-2004/064822, the disclosure of which is incorporated herein by reference. This aspect may be put into effect by treating a patient receiving a cPLA₂-alpha inhibitor therapy with a 5-lipoxygenase inhibitor(or vice versa) and preferably by co-administration or sequential administration.

In a still further aspect of the invention, a 5-lipoxygenase inhibitor may be used in the treatment of cancer in combination with a deoxycholate. This aspect of the invention finds particular application in the treatment of colon cancer. Preferably, the 5-lipoxygenase is a hydroxylamine or derivative, more preferably a sulfonamine-containing ‘reverse’ hydroxylamine, and may be any compound according to the Formulae I, II, III and IV as defined above. This aspect may be put into effect by treating a patient receiving a deoxycholate with a 5-lipoxygenase inhibitor (or vice versa) and preferably by co-administration or sequential administration.

In a still further aspect of the invention is the use of a 5-lipoxygenase inhibitor, selected from compounds according to any one of Formulae I, II, III or IV, in combination with a 15-lipoxygenase activator in the treatment of cancer, especially cancers in which the 5-lipoxygenase pathway is implicated, such as epithelial cell-derived cancers, or in particular pancreatic cancer, prostate cancer, bladder cancer, colon cancer and testicular cancer. Optionally, the use is achieved by co-administration of the 5-lipoxygenase inhibitor and the 15-lipoxygenase activator, e.g. by manufacturing a medicament comprising both components.

In a further aspect of the invention, 5-lipoxygenase inhibitors selected from those defined above according to Formulae I, II, III or IV may be used in combination therapy for the treatment of cancer with NSAIDs, optionally by co-administration or in a single therapeutic treatment.

According to a further aspect, the invention provides the use of the above defined compounds or combinations of therapies in the manufacture of a medicament for the treatment or prophylaxis of cancer, more particularly a cancer in which 5-lipoxygenase (and optionally also the further therapeutic targets referred to above) is implicated, by inhibition of 5-lipoxygenase. The disease in which 5-lipoxygenase is implicated may be, for example selected from the cancers mentioned above and may be, for example, one or more of breast cancer, colon cancer, colorectal cancer, pancreatic cancer, esophageal cancer, glioma, lung cancer including non-small cell lung cancer, prostate cancer, bladder cancer, thoracic cancer, ovarian cancer, cervical cancer, testicular cancer, renal cancer, Rubinstein-Taybi syndrome, acute promyelocytic leukaemia, acute myelogenous leukaemia and non-Hodgekins lymphoma. There is further provided a use of a compound as defined in Formulae I and II and optionally III and IV above and/or a second anti-cancer drug in the manufacture of a medicament comprising the compound of Formulae I or II and said second anti-cancer drug for the treatment of cancer by a combination or dual mechanism therapy, said second anti-cancer drug being preferably selected from for example, platinum drugs such as cis-platin, alkylating agents such as chlorambucil or temozolomide, topoisomerase inhibitors such as the Topo II inhibitor etoposide, kinase cdk inhibitors such as flavopiridol or roscovitine, bcr-abl kinase inhibitors such as Glivec®, hsp 90 inhibitors, telomerase inhibitors and/or carbamylating agents, mitoxantron, Vinca alkaloids such as vincristine and vinblastine, anthracycline antibiotics, taxanes such as paclitaxel, antifolates such as methotrexate and camptothecins such as irinotecan, but preferably from HDAC inhibitors, deoxycholate, cPLA₂-alpha inhibitors, 15-lipoxygenase activators and NSAIDs having anti-cancer properties.

Compounds described herein, and preferred compounds, especially those of formula II above, are particularly effective in the treatment of 5-lipoxygenase mediated cancers, it is believed, and without being bound by theory, because the ‘reverse’ hydroxamic acid classes described herein surprisingly have very good 5-lipoxygenase inhibitory activity, surprisingly well retained whilst having improved duration of action in vivo as a result of reduced rate of metabolism. Furthermore, the alpha-methylene substituent provides improved duration of action, whilst the preferred linker groups and their arrangement provide surprisingly improved activity and duration of action.

The invention will now be described in more detail, without limitation, with reference to the Examples.

### EXAMPLES

The following examples are provided as an illustration of the preparation of the compounds of the invention and are non-limiting.

### Example 1

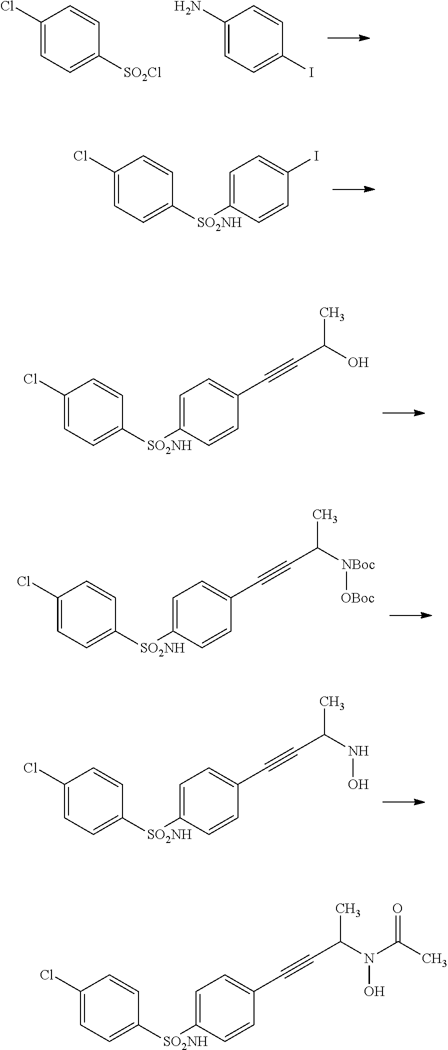

4-Iodoaniline (11 g) was dissolved in dichloroethane (150 ml). Triethylamine (20 ml) was added and the mixture cooled to 0-5° C. 4-Chlorobenzensulphonyl chloride (16 g) was added in potions over 1 h. After a further 1 h, the mixture was washed with dilute hydrochloric acid, the DCE layer separated and dried. After removal of the solvent and treating the residue with isopropyl ether, 16.5 g coupled product was obtained.

The iodide (7.9 g) was mixed with butyn-2-ol (2 g), copper (I) iodide (250 mg), tetra-kis-triphenyl phosphine Pd (0) (0.5 g) and ethyl acetate (40 ml) under nitrogen. Triethylamine (6 ml) was added, during which time the solids dissolved and there was an exotherm. After 1 h (complete reaction) the mixture was washed with dilute HCl and the solution was dried over magnesium sulphate. After filtration (also removes Cu salts), the solvent was removed and the crude product triturated with isopropyl ether to give the product (6.5 g).

The alcohol (3 g) was dissolved in dichloromethane (25 ml) together with bis-BOC hydroxylamine (2.65 g) and triphenylphosphine (2.9 g). After cooling in an ice bath, di-isopropyl azocarboxylate (2.4 g) was added dropwise. After 2 h, the solvent was removed and the residue treated with 10% toluene in hexane. After adding a trace of silica gel, the phosphine oxide:hydrazine complex crystallised and was then filtered. The residue was purified by chromatography (1:1 DCM:hexane then 2:1) to give 2.3 g product.

The bis-BOC product (1.1 g) was dissolved in 5 ml DCM and 2.5 ml trifluoroacetic acid added. After 3 h, the mixture was poured on to sodium bicarbonate/water. The hydroxylamine was extracted with DCM and dried. After removal of the solvent, the residue was treated with isopropyl ether to give the hydroxylamine (mpt, 155-156° C.).

The hydroxylamine (650 mg) was treated with 2 equivalents of acetyl chloride in pyridine (3 ml) and dichloromethane (5 ml). After 3 h, the mixture was diluted with dichloromethane and washed with dilute HCl. After drying, the residue was dissolved in methanol (10 ml) and treated with potassium carbonate (0.5 g). After 1 h, the solvent was removed, dilute HCl added and the residue isolated into dichloromethane. After drying and concentration to low volume, the product was filtered to afford 380 mg, mpt 173-174.

The 3-acetylenes may be similarly prepared.

The compounds may also be prepared by coupling with the bis-Boc acetylene.

### Example 2

The olefinic compounds may be prepared as shown below:

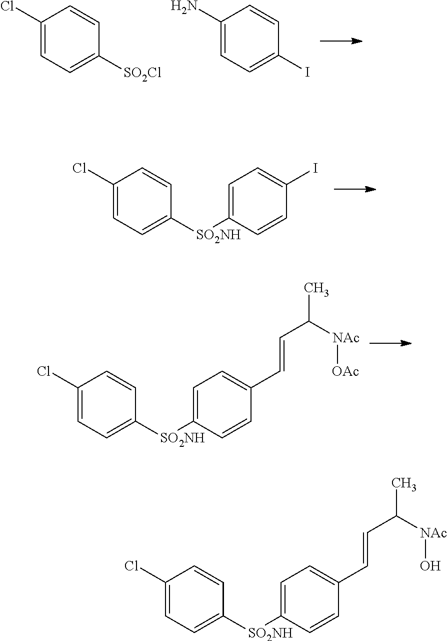

The iodide (4 g), triethylamine (2.5 ml), palladium acetate (230 mg), triphenyl phosphine (0.52 g) and the olefin (2.5 g-prepared by a Mitsunobu reaction between the alcohol and bis-acetyl hydroxylamine) were dissolved in acetonitrile (15 ml) and DMF (4 ml) and warmed to reflux for 4 h. The solvent was removed and replaced by toluene (20 ml). After washing with dilute HCl, the toluene was removed and replaced with methanol (10 ml). Sodium hydroxide (1 ml, 18M) was added and the mixture stirred for 1h. The methanol was removed, water added and the aqueous washed with diethyl ether. After acidification, the aqueous layer was extracted with DCM. After drying, the solvent was removed and the residue purified by chromatography (ethyl acetate) to give 62 mg product as a glass.

### Example 3

The following experimental sets out a procedure, as a non-limiting illustration, for preparing ‘reverse sulfonamide’ (relative to Examples 1 and 2) linked (L¹) acetylene linked (L²) hydroxamic acid derivatives according to the present invention.

### Stage 1

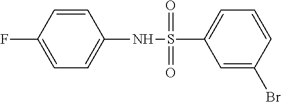

### 3-bromo-N-(4-fluorophenyl)benzenesulfonamide

3-Bromobenzene sulphonyl chloride (25.5 g, 0.1 mole) is dissolved in dichloromethane (150 ml) and sodium bicarbonate added (17 g, 0.2 mole). 4-Fluoroaniline (22.6 g, 0.2 mole) is added and the mixture stirred overnight. Water is added and the DCM phase separated, washed twice with 100 ml 3M HCl. The solution is dried and the solvent removed to afford the crude sulphonamide in essentially quantitative yield.

### Stage 2

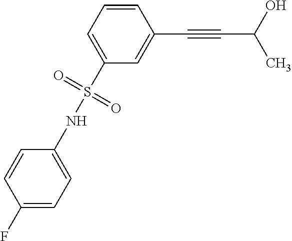

### N-(4-fluorophenyl)-3-(3-hydroxybut-1-yn-1-yl)benzenesulfonamide

The crude stage 1 product is dissolved in DMF (250 ml) and copper (I) iodide (1 g) is added. Triethylamine (21 ml, 1.5 eq) is added followed by 3-butyn-2-ol (11 ml, 1.5 eq). The mixture is warmed to 80° C. under nitrogen for 0.5 h then cooled to 50° C. and bis(acetonitrile) palladium (II) chloride (1.3 g, 5%) added. The mixture is then heated at 80° C. for approximately 6 h or until the bromide is consumed (tlc analysis). The mixture is quenched with 1 litre water and the product extracted with 3×150 ml toluene. The combined toluene fractions are washed with water. The solvent is removed and the product used without further purification.

### Stage 3

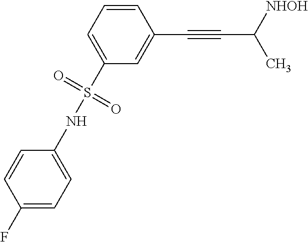

### N-(4-fluorophenyl)-3-[3-(hydroxyamino)but-1-yn-1-yl]benzenesulfonamide

The crude stage 2 product is dissolved in dichloromethane (400 ml). Pyridine (16 ml, 0.2 eq) and DMAP (200 mg) are added and the mixture cooled to 0° C. Methane sulphonyl chloride (10 ml, 1.25 eq) is added dropwise over about 0.5 h and the mixture is allowed to warm to room temperature until the reaction is complete (approximately 4 h). The mixture is washed with 2M HCl (2×200 ml) and water. The solvent is removed and the crude product dissolved in NMP (200 ml). Aqueous hydroxylamine solution is added (30 ml, 15 g, 4.5 eq) and the mixture stirred for 4 h. 1.5 litres of water were added and the product extracted with dichloromethane (2×200 ml). The dichloromethane layer is washed twice with water and dried. After removal of the drying agent, the solution is used directly in the next reaction.

### Stage 4

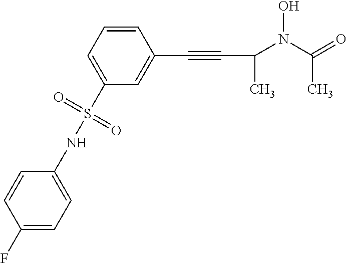

### N-(3-{3-[(4-fluorophenyl)sulfamoyl]phenyl}-1-methylprop-2-yn-1-yl)-N-hydroxyacetamide

The solution from stage 3 is mixed with pyridine (25 ml) and cooled in an ice bath. Acetyl chloride (10 ml) is added dropwise over about 15 mins. The mixture is stirred at room temperature for 2 h. 2M Hydrochloric acid (150 ml) is added and the organic phase separated, washed with water and dried. After removal of solvent, the crude product is dissolved in 200 ml methanol and treated with potassium carbonate (1 g). After 2 h, the methanol is removed, the product extracted into dichloromethane (200 ml) and washed with water. After drying and removal of solvent, the product is passed through a short silica column eluting with ethyl acetate/hexane. The isloated product is crystallised from ethyl acetate/hexane. HPLC purity >99%.

The invention has been described with reference to a preferred embodiment. However, it will be appreciated that variations and modifications can be effected by a person of ordinary skill in the art without departing from the scope of the invention.

1. A compound for use in the inhibition of 5-lipoxygenase for the treatment or prophylaxis of cancer, which compound is defined according to Formula I
Ar¹-L¹-Ar²-L²-C(R³)(R⁴)N(OR¹)C(═Y)—R² (I)
where
Y is selected from O or S
R¹ is H, a salt or readily hydrolysable substituent;
R² is selected from H or CH₃, CH₂F, CF₂H or CF₃;
R³ and R⁴ are selected independently from H, C1-4 alkyl or alkenyl, CF₃, CH₂F, CF₂H and F, with the proviso that if either R³ or R⁴ is H, then the other is not H;
L¹ is a linker group;
L² is a linker group comprising an optionally substituted or unsubstituted unsaturated branched or straight chain alkyl group;
Ar¹ is an optionally substituted or unsubstituted aryl or heterocyclic group; and
Ar² is an optionally substituted or unsubstituted aryl or heterocyclic group.

2. A compound according to claim 1, wherein R² is methyl.

3. A compound according to claim 1, wherein R³ is H and R⁴ is methyl.

4. A compound according to claim 1, wherein L¹ is selected from CH₂O, OCH₂, CH₂, CONH, NHCO, O, S, SO₂NH and NHSO₂.

5. A compound according to claim 1, wherein Ar¹ and Ar² are optionally substituted or unsubstituted phenyl groups.

6. A compound of the formula (II)
Ar¹-L¹-Ar²-L²-C(R³)(R⁴)N(OR¹)C(═Y)—R² (II)
where
Y is selected from O or S;
R¹ is H, a salt or readily hydrolysable substituent;
R² is selected from H or CH₃, CH₂F, CF₂H or CF₃;
R³ and R⁴ are selected independently from H, C1-4 alkyl or alkenyl, CF₃, CH₂F, CF₂H and F, with the proviso that if either R³ or R⁴ is H, then the other is not H;
L¹ is NHSO₂, SO₂NH, NHCONH, SO₂N—CH₂—, -(L³)ₙ-SO₂NH-(L⁴)ₘ- or (L³)ₙ-NHSO₂-(L⁴)ₘ where n and m are 0 or 1 and L³ and L⁴ are selected from CH₂ and branched or straight-chain C2-4 alkyl or alkenyl, or —SO₂— provided that where L¹ is —SO₂— then Ar¹ or Ar² is bound to L¹ via a ring nitrogen;
L² is an unsaturated C2-6 optionally substituted or unsubstituted branched or straight chain alkyl group;
Ar¹ is an optionally substituted or unsubstituted aryl or heterocyclic group; and
Ar² is an optionally substituted or unsubstituted aryl or heterocyclic group.

7. A compound according to claim 6, wherein L¹ is NHCONH, SO₂NCH₂—, —SO₂—, provided that where L¹ is —SO₂— then Ar¹ or Ar² is bound to L¹ via a ring nitrogen; or L¹ is -(L³)ₙ-SO₂NH-(L⁴)ₘ- or (L³)ₙ-NHSO₂-(L⁴)ₘ where n and m are independently 0 or 1 provided that (m+n) is not 0 and L³ and L⁴ are selected from CH₂ and branched or straight-chain C2-4 alkyl or alkenyl.

8. A compound according to claim 7, wherein R² is methyl.

9. A compound according to claim 6, wherein R³ is H and R⁴ is methyl.

10. A compound according to claim 6, which is further defined according to the formula (III)
Ph¹-L¹-Ph²-L²-C(R³)(R⁴)N(OR¹)C(═Y)—R² (III)
where
Y is selected from O or S;
R¹ is H, a salt or readily hydrolysable substituent;
R² is selected from H or CH₃, CH₂F, CF₂H or CF₃;
R³ and R⁴ are selected independently from H, C1-4 alkyl or alkenyl, CH₂F, CF₂H, CF₃ and F, with the proviso that both R³ and R⁴ are not H;
L¹ is NHCONH, SO₂NCH₂-, —SO₂—, provided that where L¹ is —SO₂— then Ar¹ or Ar² is bound to L¹ via a ring nitrogen; or L¹ is -(L³)ₙ-SO₂NH-(L⁴)ₘ- or (L³)ₙ-NHSO₂-(L⁴)ₘ where n and m are independently 0 or 1 provided that (m+n) is not 0 and L³ and L⁴ are selected from CH₂ and branched or straight-chain C2-4 alkyl or alkenyl;
L² is an unsaturated C2-4 optionally substituted or unsubstituted branched or straight chain alkyl group;
Ph¹ is an optionally substituted or unsubstituted phenyl group
Ph² is an optionally substituted or unsubstituted phenyl group

11. A compound according to claim 10, wherein R² is methyl.

12. A compound according to claim 10, wherein R³ is H and R⁴ is methyl.

13. A compound according to claim 10, wherein Ph² has a 1,3 or 1,4 substitution arrangement with respect to L¹ and L².

14. A compound according to claim 10, wherein Ph¹ comprises at least one substituent which is selected from F, Cl or Br.

15. A compound according to claim 14, wherein the at least one substituent of Ph¹ forms a 1,4 substitution arrangement on Ph¹ with L¹.

16. A compound according to claim 6, wherein L² is a trans ethylene group.

17. A compound according to claim 6, which is present in high purity enantiomeric form.

18. (canceled)

19. (canceled)

20. (canceled)

21. (canceled)

22. A pharmaceutical formulation comprising the compound according to claim 6 and a pharmaceutically acceptable excipient.

23. (canceled)

24. (canceled)

25. Use of a compound according to claim 1 or claim 6 in the manufacture of a medicament for the treatment or prophylaxis of cancers in which 5-lipoxygenase is implicated by inhibition of 5-lipoxygenase.

26. A use as claimed in claim 25, wherein the cancer is selected from breast cancer, colon cancer, colorectal cancer, esophageal cancer, glioma, lung cancer including non-small cell lung cancer, prostate cancer, pancreatic cancer, bladder cancer, brain cancers, thoracic cancer, ovarian cancer, cervical cancer, testicular cancer, renal cancer, Rubinstein-Taybi syndrome, acute promyelocytic leukaemia, acute myelogenous leukaemia and non-Hodgekins lymphoma.

27. A use as claimed in claim 26 wherein the cancer is selected from prostate cancer, pancreatic cancer, colon cancer, bladder cancer and testicular cancer.

28. Use of an inhibitor of 5-lipoxygenase and/or an inhibitor of HDAC in the manufacture of a medicament indicated for the treatment of cancer by combination therapy using an inhibitor of 5-lipoxygenase in combination with an inhibitor of HDAC.

29. (canceled)

30. A use according to claim 28, wherein the inhibitor of 5-lipoxygenase and/or the inhibitor of HDAC is selected from compounds comprising or derived from hydroxamic acids.

31. A use according to claim 30, wherein the inhibitor of 5-lipoxygenase and/or the inhibitor of HDAC is selected from compounds as defined according to Formula I of claim 1 or Formula II of claim 6.

32. (canceled)

33. (canceled)

34. (canceled)

35. (canceled)

36. (canceled)

37. (canceled)

38. (canceled)

39. A use according to claim 28, wherein the cancer is selected from breast cancer, colon cancer, colorectal cancer, esophageal cancer, glioma, lung cancer including non-small cell lung cancer, prostate cancer, pancreatic cancer, bladder cancer, brain cancers, thoracic cancer, ovarian cancer, cervical cancer, testicular cancer, renal cancer, Rubinstein-Taybi syndrome, acute promyelocytic leukaemia, acute myelogenous leukaemia and non-Hodgekins lymphoma.

40. (canceled)

41. (canceled)

42. A method for the treatment or prophylaxis of cancer in the human or animal body comprising administering to a patient in need thereof a therapeutically effective amount of 5-lipoxygenase inhibitor in order to disrupt 5-lipoxygenase activity, the 5-lipoxygenase inhibitor being selected from compounds according to claim 1 or claim 6.
